(19) United States
(12) Patent Application Publication
(10) Pub. No.: US 2023/0243824 A1
(43) Pub. Date: Aug. 3, 2023

(54) TESTING METHOD AND APPARATUS

(71) Applicant: MOLENDOTECH LIMITED, Devon (GB)

(72) Inventors: Simon Jackson, Devon (GB); Anas Sattar, Devon (GB); Christian Good, Devon (GB); Wondwossen Abate, Devon (GB)

(21) Appl. No.: 18/009,879

(22) PCT Filed: Jun. 16, 2021
(86) PCT No.: PCT/GB2021/051526

§ 371 Date: Dec. 12, 2022

(30) Foreign Application Priority Data
Jun. 16, 2020 (GB) 2009143.5

(51) Int. Cl. G01N 33/569; G01N 33/543; G01N 33/579; C12Q 1/04

(52) U.S. Cl. CPC G01N 33/56911; C12Q 1/045; G01N 33/579; G01N 33/54326; G01N 33/56961; G01N 2333/195; G01N 2400/50

(57) ABSTRACT

A testing method is described for use in undertaking a test upon a sample (10) for the presence of a specific microbiological material, the method comprising the steps of combining the sample (10) with a viability-preserving medium, optionally with an enrichment medium (12) selected to promote growth and/or reproduction of the specific microbiological material within the sample (10) and incubating the sample (10) and enrichment material (12), undertaking a separation process to separate the live specific microbiological material from the sample (10), and testing the separated material for the presence of the specific microbiological material. An apparatus for use in the method is also described.

This invention relates to a method of conducting a test upon a sample, for example a liquid sample, to ascertain whether the sample contains a specific microbiological material, and to an apparatus for use with the method.

A number of testing methodologies are known for use in testing for the presence of specific microbiological materials within samples and for enumerating the level of such microbiological contamination of the samples. By way of example, a large number of testing methodologies are known for use in laboratories to ascertain whether samples taken from, for example, hospital patients contain specific microbiological materials. Similarly, a number of tests are known and are in widespread use to test for the presence of contaminants in food or beverage preparation or production facilities and/or products produced therein. These represent just a couple of examples of applications in which such tests are undertaken.

One commonly preferred testing methodology for detecting and enumerating microbial contamination involves microbial culture. Typically, such a methodology is slow, often taking between 2 days and several weeks for the culture to be of a form or in a condition sufficient to determine a valid result, and it is a labour intensive, time consuming process. Furthermore, contamination during the testing procedure can result in the test results being inaccurate, and some materials are unable to be detected in this manner as a result of certain organisms being of a non-culturable form.

As mentioned above, one disadvantage common to many known testing methodologies is that the tests are often relatively lengthy and so there is a considerable delay between the time at which a sample is taken and the time at which the test results are available. This delay can lead to significant problems. By way of example, in food or beverage preparation or production facilities, by the time a test result issues indicating that there is a contamination issue, the food or beverage may have already been consumed or may have been delivered to customer leading to the need to recall products. Where the test is undertaken in a hospital environment or the like, the delay may result in delays to treatment of a patient, which may in turn result in worsening of a patient’s condition. Obviously, it would be desirable to provide a testing methodology by which at least some of these delays can be reduced.

According to the present invention there is provided a testing method for use in undertaking a test upon a sample for the presence of a specific microbiological material, the method comprising the steps of:
combining the sample with a viability-preserving medium selected to keep the microbiological material viable;
undertaking a selection process to select the specific microbiological material; and
testing the selected material for the presence of the specific microbiological material.

It will be appreciated that, by keeping a sample in a preserving medium, it can be ensured that the microbiological material remains present in sufficient quantity, alive, until the time it is tested. This provides a degree of confidence that the material subsequently tested was alive at the time of sampling, i.e. live microbiological material. The viability-preserving medium may be a sample-preserving medium. The viability-preserving medium may be a medium that is compatible with sustaining cells of the microbiological material, e.g. bacterial cell walls, to preserve the microbiological material in a live, or viable, condition. For instance, the sample-preserving medium may be a phosphate-buffered saline solution (PBS). The purpose of a viability-preserving medium may be to provide osmotic conditions that are not harmful to the microbiological material. The viability-preserving medium may be selected to maintain viability for a certain amount of time until the selection and/or testing steps are carried out.

It will be appreciated the sample may be collected using an appropriate equipment. The equipment may be a swab or sheath. The equipment is understood to be sterile, so as to avoid contamination with unknown agents. Likewise, the viability-preserving medium is preferably sterile.

According to an embodiment of the present invention there is provided a testing method for use in undertaking a test upon a sample for the presence of a specific microbiological material, the method comprising the steps of:
combining the sample with an enrichment medium selected to promote growth and/or reproduction of the specific microbiological material within the sample;
incubating the sample and enrichment material;
undertaking a selection process to select the specific microbiological material; and
testing the selected material for the presence of the specific microbiological material.

It will be appreciated that the method of the embodiment is advantageous in that, by using an enrichment medium that is selected to promote the growth and/or reproduction of the specific microbiological material, and then undertaking an incubation step, the presence of the specific microbiological material within the sample is magnified or enhanced. Subsequent detection of the presence of the specific microbiological material within the sample, and enumeration thereof, is thus simplified.

In some embodiments, the enrichment medium is of a composition that encourages a log-phase growth of the microbiological material.

As an alternative to, or in addition to combining the sample with an enrichment medium, the sample may be kept in a viability-preserving medium sufficient to keep the sample viable. The sample may be constituted by or comprise a colony of microbiological material, or a so-called biofilm. Such biofilms, or colonies, often provide a robust matrix for microorganisms, such as a polysaccharide matrix, providing protection from the environment, including shielding against diffusion of chemical agents and hindering mechanical removal.

Such a sample may be taken as a swap from a surface, and may comprise a relatively high count of microorganisms. It will be appreciated that at the time of taking a swab, the identity, or even the presence or absence of a particular microbiological material, may not be ascertainable from inspection of the swab alone.

The microorganisms in such colonies may be relatively robust and may not require, or may not even benefit from, a medium to promote growth. In that case, it may suffice to provide a medium that preserves the microbiological material sufficiently to keep it viable. For instance, the medium may be a saline solution (e.g. phosphate-buffered saline, PBS) of appropriate osmotic properties. The medium may otherwise contain no nutrients. Alternatively, the medium may contain nutrients. In some embodiments, the nutrients are provided as a composition promoting log phase growth. It will be understood that the composition may be chosen for a particular target microorganism.

The selection step may involve a separation step to separate the live specific microbiological material from the sample. Alternatively, the selection step may otherwise select and identify or mark the selected material.

The separation step may be undertaken using a separation material that detects the presence of the specific microbiological material, and attaches thereto. By way of example, it may comprise a suitable antibody material. The separation material is preferably provided upon magnetic beads so that a magnet can be used to separate the specific microbiological material from the remainder of the sample. In one example embodiment, a suitable immunomagnetic bead may be used. The magnetic beads are understood to be magnetic particles, such as microspheres, that in the field are referred to as ‘beads’. Such magnetic beads can be coupled with proteins such as antibody structures that are specific to a target analyte such as a target epitope of a microorganism. By way of being bound to the magnetic beads, the target analyte is thereby magnetically attractable. The target analyte may be a structure such as a cell wall protein or saccharide of a target microorganism. Thereby, a selection step is carried out as part of the isolation process of the microbiological material.

Preferably, magnetic separation is undertaken using a carrier comprising a transfer structure with a temporary magnetic field. The temporary magnetic field is understood to be a magnetic field that can be controlled to be activated and/or deactivated. It will be understood that the application and removal of a magnetic field to the transfer structure may be provided in one or more ways. One option is a controllable temporary magnetic structure such as electromagnetic arrangement. One option is a moveable arrangement of a permanent magnet structure, such as a magnetic rod, disc, beads, or other suitable elements, that can be moved so as to be more proximate or more distal to the transfer structure. The transfer structure may be removable from the magnetic material, or may be permanently attached to it.

The magnetic material may be spaced from the transfer structure while extending a magnetic field beyond the transfer structure. For instance, the carrier may be a test tube into which a magnetic material such as magnetic rod in inserted. It will be appreciated that the magnetic material is chosen of a strength such that its magnetic field extends beyond the outer walls of the test tube, at least beyond the outer walls of the base of the test tube.

To collect the biological material bound to magnetic beads from a solution, the base of the test tube is immersed into the solution, and the magnetic beads will attach, by way of the magnetic field exerted by the magnetic structure inside it, to the outer wall of test tube. The effect is such that predominantly magnetically attractable material, such as microbiological material tagged with appropriate antibodies, attaches to the outside of the test tube wall, while other material that is not tagged is more likely to remain in solution.

The magnetically attractable material can then be transferred by removing the test tube from the solution and immersing its end into another solution or bath. The magnetically attractable material can then be removed from the outside wall of the test tube. Removal from the test tube may comprise rinsing, washing, scraping, or other appropriate methods. The magnetic material may be removed or inactivated. For instance, using the example of a test tube with a magnetic rod, the magnetic rod may simply be pulled out for later re-use, whereby the magnetic field is no longer present to hold the magnetically attractable material onto the test tube outer wall. The insertion or removal of the rod may be manual, although it will be appreciated that such a design renders itself to machine controlled actuation.

Using a transfer structure (such as a test tube) with a controllable, temporary magnetic field (being temporary by way of removal of the magnet from the test tube) provides that the microbiological material can be removed from the transfer structure easier than would otherwise be the case if the material was attached directly to a magnetic component. The transfer material can be selected for transfer properties, such as inert composition, robustness, size, surface area and surface smoothness (or roughness). The magnetic material can be selected for its preferred properties, e.g. magnetic strength or otherwise. It will be understood that the transfer structure itself is preferably not susceptible to magnetic fields and may be antimagnetic. This is to avoid that repeated use of the transfer structure with a temporarily activated magnetic field magnetises the transfer structure. The transfer structure may be constituted by, or comprise, a single-use or disposable item, for instance a sheath.

The carry-over method using a temporary magnetic field allows stronger magnetic fields to be used than might otherwise be the case. Alternatively or in addition, the use of a temporary magnetic field allows the exposure time of the transfer structure in the medium to be reduced, which in turn reduces the likelihood of non-specific attachment of non-magnetic biological material also present in the solution. Thereby, a further improvement in specificity may be achieved during the carry-over phase.

Magnetic separation represents just one example of a suitable separation technique for use in the separation step, and other suitable processes may be used. Depending upon the nature of the sample and the specific microbiological material being tested for, the separation step may be undertaken using a filtration process or the like. Alternatively, a centrifugation technique could be used. It will be appreciated that these are merely examples of separation techniques that may be used, and that the selection step could take a wide range of other forms.

The testing step may be undertaken using a detection method suitable for detecting the presence of lipopolysaccharide (LPS). By way of example, the detection method may be LAL or TAL based. Other detection methods detecting the presence of, for example, lipoteichoic acid or peptidoglycan may be used, if desired, or beta glucan based techniques may be used to detect the presence of fungal organisms. It will be appreciated, therefore, that the testing method may be used in the detection of either Gram-negative or Gram-positive bacteria or in the detection of fungi. The use of such detection methods is advantageous in that they can produce results relatively quickly and accurately. As the accuracy of the results of such detection methods can be impacted by the presence of non-viable materials within the sample, the use of the separation step prior to undertaking the testing step can result in the testing method being of enhanced accuracy by separating only the live material from the sample, and the use of the selective enrichment process — if carried out — will further enhance testing accuracy.

The method of the invention may be undertaken relatively quickly. By way of example, the incubation step may be of duration in the range of 15 minutes to 24 hours, preferably in the region of 2-8 hours, and so the testing method as a whole may be undertaken and produce results in less than 10 hours. Depending upon the nature of the microbiological material for the presence of which the test is being conducted, the incubation step, and hence the overall time taken to undertake the test, may be considerably shorter than this. By way of example it is envisaged that testing for the presence of E. coli could be undertaken in less than 5 hours. The testing could be undertaken outside of a conventional laboratory environment, if desired.

It will be appreciated, therefore, that the testing method of the present invention is advantageous in that accurate, reliable test results, testing for the presence of a specific microbiological material within a sample, and enumeration thereof, can be achieved more quickly and efficiently than is typically the case.

Where the specific microbiological material is E. coli O157, the enrichment medium may comprise a modified tryptone soy broth supplemented with antibiotics and/or other selective media to positively select and enhance the growth and/or reproduction of the specific microbiological material. The incubation step may be undertaken for a period in the range of 15 minutes to 24 hours, preferably 2-8 hours, and may be undertaken at a temperature of 5 to 50° C., preferably 10 to 45° C., and more preferably 20-40° C. It is preferably undertaken at a temperature of approximately 37° C., and is preferably undertaken for a duration of approximately 4 hours. However shorter incubation durations (such as 2-4 hours) or longer incubation durations (such as 5-8 hours) may be employed, if desired.

After addition of the separation material, a further incubation step may be undertaken. By way of example, it may be for a duration of 2-20 minutes, preferably 5-15 minutes, and is more preferably about 10 minutes in length.

A washing step is preferably undertaken before undertaking the testing step. By way of example, a small volume of a suitable wash buffer material such as a suitable phosphate buffer may be used.

The invention further relates to an apparatus for use in the method described hereinbefore.

The method disclosed herein is advantageous in that it detects microbiological material already present on live microorganisms, such as LPS endotoxins, after a separation step to provide a high level of specificity of the testing step. Thereby, the method provides an increased likelihood, and practically ensures, that the testing is highly specific even though the microbiological material may be otherwise ubiquitous.

The method therefore does not rely on the reproduction of genetic material in sufficient quantities, as is the case, for instance, with polymerase chain reaction (PCR) type methods. While PCR methods can achieve high specificity, these require some time in the region of many hours, typically a day or more, for their execution.

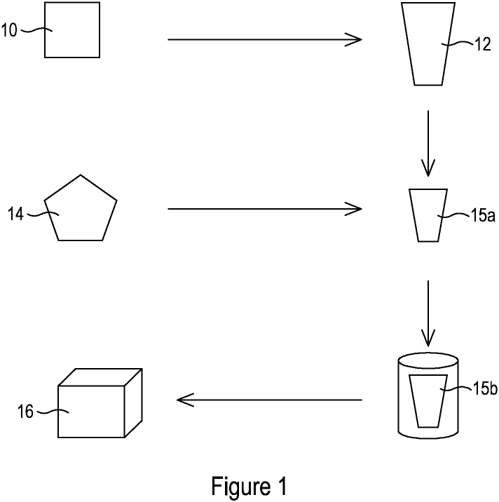

The invention will further be described, by way of example, with reference to the accompanying drawing, FIG. 1, which is a diagram illustrating the steps of a testing method in accordance with an embodiment of the invention.

Referring to FIG. 1, a testing method in accordance with an embodiment of the invention comprises the steps of acquiring a sample 10, for example of liquid form or liquefied form, of a material to be tested for the presence of a specific microbiological material, in this case E. coli O157. The sample 10 is combined and mixed with an enrichment medium 12 which is selected to enhance the growth and/or reproduction of the specific microbiological material within the sample 10. By way of example, the enrichment medium 12 may take the form of a modified tryptone soy broth which has been supplemented with antibiotics or other selective media so that the growth and/or reproduction of the specific microbiological material is enhanced, but the growth and/or reproduction of other microbiological materials is unaffected or is discouraged. Once the sample 10 and enrichment medium 12 have been combined, the sample 10 and enrichment medium 12 are placed within an incubator for an incubation period of, preferably, 4 hours, the incubator being set to incubate the sample 10 and enrichment medium 12 at a temperature of 37° C. Whilst this specific temperature and duration is suitable for use in testing for the presence of E. coli O157, it will be appreciated that where the test is for the presence of other materials, or where other enrichment media are used, then it may be preferred to incubate for a longer or shorter period of time, and/or for different incubation temperatures to be used. By way of example, incubation may be at a temperature in the range of 20-40° C., and for a duration in the range of 2-8 hours.

After incubation, it will be appreciated that if the sample initially contained a quantity of the specific microbiological material, the enrichment and incubation steps will have magnified or increased the concentration of the specific microbiological material in the sample, making detection thereof simpler.

As set out before, in alternative application scenarios, depending on the type of sample, an enrichment and incubation step may not be necessary or beneficial. Without wishing to be bound by theory, it is believed that particularly biofilm material, such as surface colonies of microorganisms, are often already present in relatively high density, and/or are shielded from external chemicals by a polysaccharide matrix. Such microorganisms can be relatively robust, such that it may suffice to merely ensure that the sample collected for subsequent testing remains viable. The sample may be held in a medium that preserves the sample in a viable condition, such as for example phosphate-buffered saline that provides a suitable osmotic environment for a microorganism but is otherwise not designed to promote growth. Any other suitable sample-preserving medium may be used.

Tests using biofilm material were carried out using test metal discs provided with reference biofilm cultures. Material from such metal discs was harvested using sterile swabs, and transferred into a sterile medium (phosphate-buffered saline), constituting a viability-preserving medium, and vortexed. The microbiological material thus harvested may be present in sufficiently large density (cell count) that no enrichment step is required. Alternatively, an enrichment step in a suitable design growth medium may be carried out to increase the cell count and/or as a further confirmation that the microbiological material in the sample was capable of growing (reproducing).

After the collection of the material, and/or after incubation, a selection step in the form of a separation step is undertaken in order to select and separate live microbiological material of the specific form from the sample 10. Whilst a range of separation techniques could be used, in this embodiment of the invention the separation is undertaken by adding an antibody material 14 labelled for interaction with the specific microbiological material, the antibody material being attached to magnetic beads, to the sample 10 and enrichment medium 12. The antibody material 14 conveniently takes the form of a suitable immunomagnetic bead material. Once the antibody material 14 has been added, a further short incubation step 15a is undertaken to allow the antibody material 14 to interact or bind with the specific microbiological material contained within sample 10. By way of example, the further short incubation step may be of duration in the range of 2-20 minutes, preferably in the range of 5-15 minutes, and is conveniently in the region of 10 minutes. It will be understood that the either the antibody material 14 is added to the microbiological material or alternatively microbiological material is added to the antibody material.

After the further short incubation period, in a step 15b a magnet is used to separate the antibody material 14, and the specific microbiological material that has become bound thereto or associated therewith, from the sample 10, and a washing step is undertaken, for example using a small amount of a suitable wash buffer such as a phosphate buffer, and the washed material may be suspended in a further quantity of the wash buffer material.

When using a magnet to separate antibody material 14, the magnetic separation may be carried out by a magnetic field provided temporarily on a transfer structure of a carrier. In one embodiment, the carrier is provided by a test tube and the temporary magnetic field is provided by a magnetic rod insertable into and removable from the test tube, to temporarily provide a magnetic field at the outer surface of the end of the test tube. The test tube is understood to be of glass or polymer material that is not magnetised by the magnetic rod. Other suitable materials may be used.

It will be appreciated that the separated material, once washed, consists substantially exclusively of the specific microbiological material (if present in the original sample) and the antibody material bound thereto as part of the separation step. If the specific microbiological material were present in the original sample, then it will be present in relatively high concentration within the washed, separated material and so should be relatively easy to detect. Conversely, if target microbiological material was not present in relevant quantities, then the separation medium will not contain microbiological material. For example, if magnetic beads are used as the separation medium, the magnetic beads will be transferred, using the above-described magnetic transfer, without the antibody-bound microbiological material.

Once washed, a test step 16 is undertaken on the washed material, to identify whether the washed material contains biological material. By way of example, where the testing method is being used to test for E. coli O157 or other Gram-negative biological material then the test step 16 may involve the use of a test sensitive to the presence of LPS. By way of example, the test may be a LAL or TAL based test. However, where the microbiological material that the testing method is being used to detect is of a Gram-positive form, for example staphylococcus, listeriae or fungal materials, then the test step 16 may be of a different form, for example testing for the presence of lipoteichoic acid or peptidoglycan. The test step 16 is preferably undertaken using a test that produces an output not only indicative of the presence of the material under test, but also allowing enumeration of the level of contamination. As the level of contamination within the sample is enhanced through the use of the enrichment medium, it will be appreciated that some adjustment of the level of contamination indicated by the result of the test step 16 may need to be made to provide an accurate indication of the level of contamination of the sample 10 with the microbiological material under test.

Due to the selective nature of the separation step, a non-result provides a high level of confidence that no target microbiological material was present.

Conducting TAL or LAL based tests, for example of the gel clotting or colour change form, to identify whether LPS is present in the separated material, and using this as a marker for the presence of the specific microbiological material within the sample, and the approximate level thereof, may be undertaken in a simple and conveniently manner, such tests being well known and simple to use. Conveniently, the washed, separated material is placed within a series of sterile tubes or the like, in which the tests are undertaken. By suitably diluting the samples or using different test methods, an assessment of the concentration of the specific microbiological material can be made.

It will be appreciated that the testing method of the invention is advantageous in that testing may be undertaken in a relatively quick manner for the presence of specific microbiological materials. Limited specific equipment is required in order to undertake the tests, and so the testing method can potentially be undertaken in environments remote from laboratories, and testing may be undertaken without requiring individuals undertaking the tests to be skilled in laboratory techniques.

Whilst a specific embodiment of the invention is described herein with reference to the accompanying drawing, it will be appreciated that a wide range of modifications and alterations may be made thereto without departing from the scope of the invention as defined by the appended claims.

The invention is thought to be useful in scenarios in which a lengthy testing time in the region of several days is not appropriate or convenient. Certain embodiments of the invention are further thought to be of benefit for microorganisms that tend to be difficult to grow in traditional enrichment media, particularly microorganisms present in biofilms.

The methods disclosed herein may be used to test for the presence or absence of microbiological material, including but not exclusively biofilms, on medical devices, for human and/or veterinary use, on any surfaces thereof and particularly inside and/or luminal regions of medical devices, such as those of catheters, needles, reservoirs, tubing, connector surfaces, surgical instruments, drills, implants, dental corrective devices and dentures etc., as well as wound management, dressings, wound swabs etc.

The methods disclosed herein may be used to test for the presence or absence of microbiological material, contamination measurements and/or measurement of contamination levels, including but not exclusively biofilms, on food items, for human and animal consumption, feed, dairy products, eggs, fish, crustaceans, molluscs, plants, meat, cosmetics, as well as processing lines, packaging, bottling, and processing areas and equipment for such articles and combinations thereof. Likewise, the methods may be used for testing of farming and reproduction equipment, aquaculture, ballast water and others.

Likewise, the methods disclosed herein are thought to be useful in the testing of fuel processing, particularly biofuel processing where contamination with microorganisms can remain otherwise undetected.

The methods may be used in the form of a series and/or array of tests. For instance, test swabs of surfaces with suspected biofilm growth may be taken at spaced-apart intervals to map out the spread of a biofilm. It will be appreciated that such series of tests, repeatedly taken over a period of time, allow dynamics of biofilm development to be investigated. The method may be used to understand microbiological growth under external influences, to observe the attenuation and/or inhibition effect of anti-microbial agents, barriers, radiation etc. and different combinations and concentrations thereof. The method may therefore be used to study resistance and tolerance levels of microbiological organisms.

The methods disclosed herein may be combined and used in parallel. For instance, a sample from one source may be processed in accordance with embodiments without exposure to an enrichment medium, and another sample from the same source may be processed in accordance with embodiments comprising exposure to an enrichment medium. It will be understood that the non-enriched sample may be processed sooner, potentially instantaneously, and the enriched sample may be processed a few hours later, after an enrichment phase. Any differences between the results of a pre-enriched sample and an enriched sample may be considered in the analysis of the microbiological material.

1. A testing method for use in undertaking a test upon a sample for the presence of a specific microbiological material, the method comprising the steps of:
combining the sample with a viability-preserving medium selected to keep the microbiological material viable;
undertaking a selection process to select the specific microbiological material; and
testing the selected material for the presence of the specific microbiological material.

2. The method according to claim 1, wherein the step of combining the sample with a viability-preserving medium comprises combining the sample with an enrichment medium selected to promote one of growth and reproduction of the specific microbiological material within the sample; and incubating the sample and enrichment material.

3. The method according to claim 1, wherein the step of combining the sample with a viability-preserving medium comprises transferring the sample into a phosphate-buffered saline solution.

4. The method according to claim 1, wherein the selection process comprises a separation step in which the selected material is separated from the sample.

5. The method according to claim 4, wherein the separation step is undertaken using a separation material that detects the presence of the specific microbiological material, and attaches thereto.

6. The method according to claim 5, wherein the separation material comprises a suitable antibody material.

7. The method according to claim 5, wherein the separation material is provided upon magnetic beads so that a magnet can be used to separate the specific microbiological material from the remainder of the sample.

8. The method according to claim 4, wherein the separation step comprises at least one of a filtration process and a centrifugation process.

9. The method according to claim 1, wherein the testing step is sensitive to the presence of gram-negative material.

10. The method according to claim 9, wherein the testing step is undertaken using a detection method suitable for detecting the presence of lipopolysaccharide (LPS).

11. The method according to claim 10, wherein the detection method is one of LAL based or TAL based.

12. The method according to claim 1, wherein the testing step is sensitive to the presence of gram-positive material.

13. The method according to claim 12, wherein the detection method detects the presence of at least one of lipoteichoic acid or peptidoglycan.

14. The method according to, wherein the testing step is sensitive to the presence of fungal material.

15. The method according to claim 14, wherein the testing method is a beta-glucan based test.

16. The method according to claim 1, wherein the enrichment medium comprises a modified tryptone soy broth supplemented with at least one of antibiotics and selective media, to positively select and enhance at least one of the growth and the reproduction of the specific microbiological material.

17. The method according to claim 1, wherein the incubation step is undertaken for a period in the range of 15 minutes to 24 hours.

18. (canceled)

19. The method according to claim 1, wherein the incubation step is undertaken at a temperature of 5 to 50° C.

20. The method according to claim 1, wherein a washing step is undertaken before undertaking the testing step.

21. An apparatus adapted for use in the method of claim 1.
